(19) United States
(12) Patent Application Publication
(10) Pub. No.: US 2004/0247687 A1
(43) Pub. Date: Dec. 9, 2004

(54) Method for production of active ingredient-containing pellets

(75) Inventors: Hans-Ulrich Petereit, Darmstadt (DE); Guenter Bergmann, Gross-Krotzenburg (DE); Klaus Lehmann, Rossdorf (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: ROEHM GMBH & CO. KG, Darmstadt (DE)

(21) Appl. No.: 10/489,853

(22) PCT Filed: Oct. 16, 2002
(86) PCT No.: PCT/EP02/11556

(30) Foreign Application Priority Data
Oct. 22, 2001 (DE) 101 51 290.2

(51) Int. Cl. A61K 9/50; B29B 9/00

(52) U.S. Cl. 424/489; 264/5

(57) ABSTRACT

The invention relates to a method for the production of active ingredient-containing pellets by the steps of a) suspending or dissolving a pharmaceutical active ingredient in a dispersion, which contains a cationic copolymer, b) dropping said dispersion, containing the pharmaceutical active ingredient and the copolymer in an aqueous polymer solution, which contains an anionic copolymer, incompatible with the cationic copolymer, so that active ingredient-containing pellets precipitate and c) separating said active ingredient-containing pellets from the aqueous polymer solution and then drying said pellets. The invention also relates to said pellets and the use thereof in diagnostics or cosmetics, as well as pharmaceutical forms which contain said pellets.

The invention relates to a method for the production of active ingredient-containing pellets, and to pellets which can be produced thereby having a multilayer structure and to the use thereof.

### PRIOR ART

DE 100 13 029 describes the use of a multilayer pharmaceutical form which is essentially composed of a core with an active pharmaceutical ingredient, an inner coating of a copolymer or a mixture of copolymers which are composed of 85 to 98% by weight of free-radical polymerized C₁- to C₄-alkyl esters of acrylic or methacrylic acid and 15 to 2% by weight of (meth)acrylate monomers with a quaternary ammonium group in the alkyl radical, and an outer coating of a copolymer which is composed of 75 to 95% by weight of free-radical polymerized C₁- to C₄-alkyl esters of acrylic or methacrylic acid and 5 to 25% by weight of (meth)acrylate monomers with an anionic group in the alkyl radical.

Graf, E. and Bothe W. describe in “Mikroverkapselung durch Zertropfen” (Pharmazie in unserer Zeit, 1984, No. 3, pp. 71 to 82) in particular the microencapsulation of medicinal substances by delivering droplets from liquid jets. The production of microcapsules is indicated inter alia by the example of methacrylate copolymers with anionic radicals (EUDRAGIT® S100). For this purpose, EUDRAGIT® S100 is dissolved in a mixture of ethanol, acetone and isopropanol, and in addition a proportion of water is incorporated, and used to encapsulate olive oil. Acidified water of pH 4.0 is employed as precipitating bath. The resulting capsules have very thin walls and are unable to hold the oil. The microencapsulation of oils with EUDRAGIT® to give free-flowing powders is not regarded as being possible. The use of such capsules in aqueous liquids is, however, regarded as conceivable.

### Problem and Solution

The production of pharmaceutical forms such as tablets or pellets by coating active ingredient-containing cores with a polymeric coating agent is sufficiently well known. The intention was to provide an alternative method for the production of active ingredient-containing pellets, the intention being to avoid the use of organic solvent.

The problem is solved by a method for the production of active ingredient-containing pellets through the steps of

a) suspending or dissolving an active pharmaceutical ingredient in a dispersion which comprises a cationic copolymer,

b) delivering droplets of the dispersion comprising the active pharmaceutical ingredient and the copolymer into an aqueous polymer solution which comprises an anionic copolymer incompatible with the cationic copolymer, resulting in precipitation of active ingredient-containing pellets

c) removal of the active ingredient-containing pellets from the aqueous polymer solution and subsequent drying of the pellets.

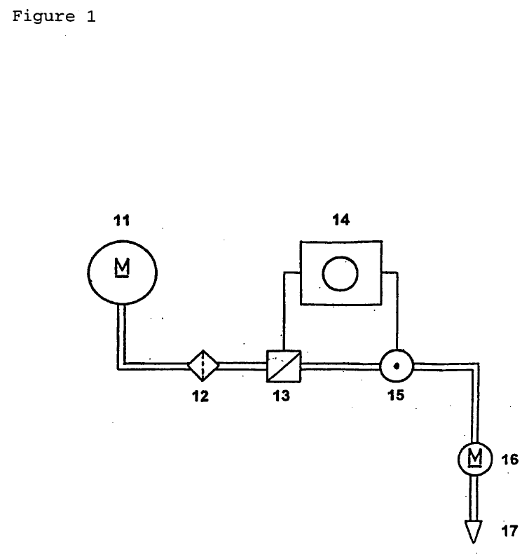

The invention is illustrated by FIGS. 1/2 and 2/2 but is not restricted to this depiction.

FIG. 1/2: diagrammatic construction of a dropping apparatus for delivering droplets of the active ingredient-containing dispersion.

Reference numbers:

11=delivery pump, pressure vessel, tubing pump or eccentric screw pump (e.g. type 6.2 from Wangen GmbH, 7988 Wangen, Germany), M=motor

12=extra fine filter 15 μm (e.g. type FW, from B.E.S.T., 6000 Frankfurt am Main 60, Germany)

13=needle valve with controllable servomotor (e.g. type SS, from B.E.S.T.)

14=PID controller with digital pressure display (e.g. type 810, from Eurotherm, 62500 Limburg, Germany)

15=pressure transducer 0 to 1.67 bar (e.g. type AB from Frey, 80000 Munich, Germany)

16=vibratory drive (e.g. type DR 40, from Retsch GmbH, 5657 Haan 1), M=motor

17=nozzle with exchangeable insert (e.g. type WAI, from Walther, 6500 Wuppertal)

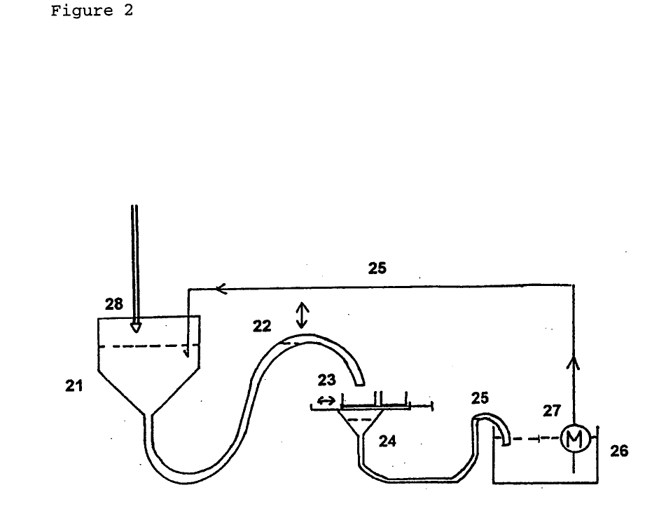

FIG. 2/2: diagrammatic construction of the precipitation bath with the solution of the anionic methacrylate copolymer

21=receiving container for precipitation bath liquid

22=flexible tubing for discharging precipitated pellets. Permits adjustment of the level of liquid in the receiving container by the siphon principle

23=exchangeable sieve for removing the precipitated pellets

24=funnel for receiving the clarified precipitation liquid

25=tubing

26=reservoir container for precipitation bath liquid

27=tubing pump to generate a constant flow (M=motor of the tubing pump)

28=nozzle of the dropping apparatus (corresponds to FIG. 1/2 (17))

### CARRYING OUT THE INVENTION

The invention relates to a method for the production of active ingredient-containing pellets by the steps of

a) suspending or dissolving an active pharmaceutical ingredient in a dispersion which comprises a cationic copolymer,

b) delivering droplets of the dispersion comprising the active pharmaceutical ingredient and the copolymer into an aqueous polymer solution which comprises an anionic copolymer incompatible with the cationic copolymer, resulting in precipitation of active ingredient-containing pellets

c) removal of the active ingredient-containing pellets from the aqueous polymer solution and subsequent drying of the pellets.

Method Step a)

Suspending or dissolving an active pharmaceutical ingredient in a dispersion which comprises a cationic copolymer. This can also be referred to as dropping liquid for simplicity.

Cationic Copolymer

A suitable cationic-copolymer consists of free-radical polymerized units of C₁- to C₄-alkyl esters of acrylic or methacrylic acid and units of (meth)acrylate monomers with tertiary or quaternary amino or ammonium groups.

The copolymer is composed of 30 to 80% by weight of free-radical polymerized C₁- to C₄-alkyl esters of acrylic or methacrylic acid and 70 to 20% by weight of (meth)acrylate monomers with a tertiary ammonium group in the alkyl radical.

Suitable monomers with functional tertiary ammonium groups are listed in U.S. Pat. No. 4,705,695, column 3, line 64 to column 4, line 13. Particular mention should be made of dimethylaminoethyl acrylate, 2-dimethylaminopropyl acrylate, dimethylaminopropyl methacrylate, dimethylaminobenzyl acrylate, dimethylaminobenzyl methacrylate, (3-dimethylamino-2,2-dimethyl)propyl acrylate, dimethylamino-2,2-dimethyl)propyl methacrylate, (3-diethylamino-2,2-dimethyl)propyl acrylate and diethylamino-2,2-dimethyl)propyl methacrylate. Dimethylaminoethyl methacrylate is particularly preferred.

The content of monomers with tertiary ammonium groups in the copolymer can advantageously be between 20 and 70% by weight, preferably between 40 and 60% by weight. The proportions of C₁- to C₄-alkyl esters of acrylic or methacrylic acid is 80-30% by weight. Mention should be made of methyl methacrylate, ethyl methacrylate, butyl methacrylate, methyl acrylate, ethyl acrylate and butyl acrylate.

A suitable (meth)acrylate copolymer with tertiary amino groups may be composed for example of 20-30% by weight of methyl methacrylate, 20-30% by weight of butyl methacrylate and 60-40% by weight of dimethylaminoethyl methacrylate.

A specifically suitable commercially available (meth)acrylate copolymer with tertiary amino groups is composed for example of 25% by weight of methyl methacrylate, 25% by weight of butyl methacrylate and 50% by weight of dimethylaminoethyl methacrylate (EUDRAGIT® E100).

EUDRAGIT® RS/RL Type

Corresponding (meth)acrylate copolymers are disclosed for example in EP-A 181 515 or DE 1 617 751. They are polymers which are soluble or swellable independently of the pH and which are suitable for pharmaceutical coatings. A possible production method to be mentioned is bulk polymerization in the presence of a free-radical initiator dissolved in the monomer mixture. The polymer can also be produced likewise by solution or precipitation polymerization. The polymer can be obtained in this way in the form of a fine powder, achievable in the case of bulk polymerization by grinding and in the case of solution and precipitation polymerization for example by spray drying.

The (meth)acrylate copolymer is composed of 85 to 98% by weight of free-radical polymerized C₁- to C₄-alkyl esters of acrylic or methacrylic acid and 15 to 2% by weight of (meth)acrylate monomers with a quaternary ammonium group in the alkyl radical.

Preferred C₁- to C₄-alkyl esters of acrylic or methacrylic acid are methyl acrylate, ethyl acrylate, butyl acrylate, butyl methacrylate and methyl methacrylate.

The particularly preferred (meth)acrylate monomer with quaternary ammonium groups is 2-trimethylammoniumethyl methacrylate chloride.

A corresponding copolymer may be composed for example of 50-70% by weight of methyl methacrylate, 20-40% by weight of ethyl acrylate and 7-2% by weight of 2-trimethylammoniumethyl methacrylate chloride.

A specifically suitable copolymer contains 65% by weight of methyl methacrylate, 30% by weight of ethyl acrylate and 5% by weight of 2-trimethylammoniumethyl methacrylate chloride be composed (EUDRAGIT® RS).

A further suitable (meth)acrylate copolymer may be composed for example of 85 to less than 93% by weight of C₁- to C₄-alkyl esters of acrylic or methacrylic acid and more than 7 to 15% by weight of (meth)acrylate monomers with a quaternary ammonium group in the alkyl radical. Such (meth)acrylate monomers are commercially available and have been used for a long time for release-slowing coatings.

A specifically suitable copolymer contains for example 60% by weight of methyl methacrylate, 30% by weight of ethyl acrylate and 10% by weight of 2-trimethylammoniumethyl methacrylate chloride (EUDRAGIT® RL).

Dispersion

The cationic (methacrylate) copolymer is in the form of an aqueous dispersion, e.g. with a solids content of from 10 to 60, preferably 20 to 40, % by weight.

To achieve specific release profiles it is possible for mixtures of said cationic methacrylate copolymers to be present in the dispersion. The mixing can take place by mixing powders and subsequently converting them into a mixed dispersion or by mixing individual dispersions in the aqueous state.

The viscosity of the polymer dispersion is preferably about the same as, or particularly preferably higher than, the viscosity of the aqueous polymer solution which is employed as precipitation bath. The viscosity of the polymer dispersion, measured by method 2.2.10 (Totating Viscometer Method) according to Pharm. Eur. 3^{rd} edition (1997) with a Brookfield rotating viscometer, can be for example from 20 to 5 000 mPa s.

The polymer dispersion may be adjusted to a temperature below room temperature.

Active Pharmaceutical Ingredient

The dispersion comprises one or more active pharmaceutical ingredients in dissolved or dispersed form. The active ingredient may be, for example, initially dissolved or dispersed in water or another suitable solvent and then added to the dispersion with the cationic methacrylate copolymer, or mixed therein.

Active Pharmaceutical Ingredients

The formulation of the invention is suitable for administering in principle any active pharmaceutical ingredients, which are preferably to be released in the intestine and/or colon, and especially those which can advantageously be administered in slow-release form, such as antidepressants, beta-receptor blockers, antidiabetics, analgesics, antiinflammatory drugs, antirheumatics, antihypotensives, antihypertensives, psychoactive drugs, tranquilizers, antiemetics, muscle relaxants, glucocorticoids, agents for the treatment of ulcerative colitis or Crohn's disease, antiallergics, antibiotics, antiepileptics, anticoagulants, antimycotics, antitussives, arteriosclerosis remedies, diuretics, enzymes, enzyme inhibitors, gout remedies, hormones and their inhibitors, cardiac glycosides, immunotherapeutics and cytokines, laxatives, lipid-lowering agents, gastrointestinal therapeutics, migraine remedies, mineral products, otologicals, Parkinson remedies, thyroid therapeutics, spasmolytics, platelet aggregation inhibitors, vitamins, cytostatics and metastasis inhibitors, phytopharmaceuticals, chemotherapeutic agents and amino acids.

Examples of suitable active ingredients are acarbose, non-steroidal antirheumatia, cardiac glycosides, acetylsalicylic acid, virustatics, aclarubicin, acyclovir, cisplatin, actinomycin, alpha- and beta-sympathomimetics, (allopurinol, alosetron, alprostadil, prostaglandins, amantadine, ambroxol, amlodipine, methotrexate, S-aminosalicylic acid, amitriptyline, amlodipine, amoxicillin, anastrozole, atenolol, atorvastatin, azathioprine, balsalazide, beclomethasone, betahistine, bezafibrate, bicalutamide, diazepam and diazepam derivatives, budesonide, bufexamac, buprenorphine, methadone, calcium salts, potassium salts, magnesium salts, candesartan, carbamazepine, captopril, cefalosporins, celetoxib, cetirizine, chenodeoxycholic acid, ursodeoxycholic acid, theophylline and theophylline derivatives, trypsins, cimetidine, clarithromycin, clavulanic acid, clindamycin, clobutinol, clonidine, cotrimoxazole, codeine, caffeine, vitamin D and derivatives of vitamin D, colestyramine, cromoglicic acid, coumarin and coumarin derivatives, cysteine, cytarabine, cyclophosphamide, ciclosporin, cyproterone, cytarabine, dapiprazole, desogestrel, desonide, dihydralazine, diltiazem, ergot alkaloids, dimenhydrinate, dimethyl sulfoxide, dimethicone, dipyridarnoi, domperidone and domperidane derivatives, donepzil, dopamine, doxazosin, doxorubicin, doxylamine, dapiprazole, benzodiazepines, diclofenac, glycoside antibiotics, desipramine, econazole, ACE inhibitors, enalapril, ephedrine, epinephrine, epoetin and epoetin derivatives, morphinans, calcium antagonists, irinotecan, modafinil, orlistat, peptide antibiotics, phenytoin, riluzoles, risedronate, sildenafil, topiramate, macrolide antibiotics, esomeprazole, estrogen and estrogen derivatives, progestogen and progestogen derivatives, testosterone and testosterone derivatives, androgen and androgen derivatives, ethenzamide, etofenamate, etofibrate, fenofibrate, etofylline, etoposide, famciclovir, famotidine, felodipine, fenofibrate, fentanyl, fenticonazole, gyrase inhibitors, fluconazole, fludarabine, flunarizine, fluorouracil, fluoxetine, flurbiprofen, ibuprofen, flutamide, fluvastatin, follitropin, formoterol, fosfomicin, furosemide, fusidic acid, galantamine, gallopamil, ganciclovir, gemfibrozil, gentamicin, ginkgo, St John's wort, glibenclamide, urea derivatives as oral antidiabetics, glucagon, glucosamine and glucosamine derivatives, glutathione, glycerol and glycerol derivatives, hypothalamus hormones, goserelin, gyrase inhibitors, guanethidine, halofantrine, haloperidol, heparin and heparin derivatives, hyaluronic acid, hydralazine, hydrochlorothiazide and hydrochlorothiazide derivatives, salicylates, hydroxyzine, idarubicin, ifosfamide, imipramine, indometacin, indoramine, insulin, interferons, iodine and iodine derivatives, isoconazole, isoprenaline, glucitol and glucitol derivatives, itraconazole, ketoconazole, ketoprofen, ketotifen, lacidipine, lansoprazole, levodopa, levomethadone, thyroid hormones, lipoic acid and lipoic acid derivatives, lisinopril, lisuride, lofepramine, lomustine, loperamide, loratadine, maprotiline, mebendazole, mebeverine, meclozine, mefenamic acid, mefloquine, meloxicam, mepindolol, meprobamate, meropenem, mesalazine, mesuximide, metamizole, metformin, methotrexate, methylphenidate, methylprednisolone, metixen, metoclopramide, metoprolol, metronidazole, mianserin, miconazole, minocycline, minoxidil, misoprostol, mitomycin, mizolastine, moexipril, morphine and morphine derivatives, evening primrose, nalbuphine, naloxone, tilidine, naproxen, narcotine, natamycin, neostigmine, nicergoline, nicethamide, nifedipine, niflumic acid, nimodipine, nimorazole, nimustine, nisoldipine, adrenaline, and adrenaline derivatives, norfloxacin, novaminsulfone, noscapine, nystatin, ofloxacin, olanzapine, olsalazine, omeprazole, omoconazole, ondansetron, orlistat, oseltamivir, oxaceprol, oxacillin, oxiconazole, oxymetazoline, pantoprazole, paracetamol, paroxetine, penciclovir, oral penicillins, pentazocine, pentifylline, pentoxifylline, perphenazine, pethidine, plant extracts, phenazone, pheniramine, barbituric acid derivatives, phenylbutazone, phenytoin, pimozide, pindolol, piperazine, piracetam, pirenzepine, piribedil, piroxicam, pramipexol, pravastatin, prazosin, procaine, promazine, propiverine, propranolol, propyphenazone, prostaglandins, protionamide, proxyphylline, quetiapine, quinapril, quinaprilate, ramipril, ranitidine, reproterol, reserpine, ribavirin, rifampicin, risperidone, ritonavir, ropinirol, rosiglitazone, roxatidine, roxithromycin, ruscogenin, rutoside and rutoside derivatives, sabadilla, salbutamol, salmeterol, scopolamine, selegiline, sertaconazole, sertindole, sertralion, silicates, simvastatin, sitosterol, sotalol, spaglumic acid, sparfloxacin, spectinomycin, spiramycin, spirapril, spironolactone, stavudine, streptomycin, sucralfate, sufentanil, sulbactam, sulfonamides, sulfasalazine, sulpiride, sultamicillin, sultiam, sumatriptan, suxamethonium chloride, tacrine, tacrolimus, taliolol, tamoxifen, taurolidine, tazarotene, tegaserod, temazepam, teniposide, tenoxicam, terazosin, terbinafine, terbutaline, terfenadine, terlipressin, tertatolol, tetracyclines, tetryzoline, theobromine, theophylline, butizine, thiamazole, phenothiazines, thiotepa, tiagabine, tiapride, propionic acid derivatives, ticlopidine, timolol, tinidazole, tioconazole, tioguanine, tioxolone, tiropramide, tizanidine, tolazoline, tolbutamide, tolcapone, tolnaftate, tolperisone, topotecan, torasemide, antiestrogens, tramadol, tramazoline, trandolapril, tranylcypromine, trapidil, trazodone, triamcinolone and triamcinolone derivatives, triamterene, trifluperidol, trifluridine, trimethoprim, trimipramine, tripelennamine, triprolidine, trifosfamide, tromantadine, trometamol, tropalpin, troxerutin, tulobuterol, tyramine, tyrothricin, urapidil, ursodeoxycholic acid, chenodeoxycholic acid, valaciclovir, valdecoxib, valproic acid, vancomycin, vecuronium chloride, venlafaxine, verapamil, vidarabine, vigabatrine, viloxazine, vinblastine, vincamine, vincristine, vindesine, vinorelbine, vinpocetine, viquidil, warfarin, xantinol nicotinate, xipamide, zafirlukast, zalcitabine, zanamivir, zidovudine, zolmitriptan, zolpidem, zoplicone, zotepine and the like.

Examples of particularly preferred active ingredients are analgesics such as tramadol or morphine, agents for the treatment of ulcerative colitis or Crohn's disease, such as 5-aminosalicylic acid, corticosteroids, such as budesonide, proton pump inhibitors such as omeprazole, virustatics such as acyclovir, lipid-lowering agents such as simvastatin or pravastatin, H2 blockers such as ranitidine or famotidine, antibiotics such as amoxicillin and/or clavulanic acid, and ACE inhibitors such as enalapril or amlodipine.

The active ingredients can, if desired, also be used in the form of their pharmaceutically acceptable salts or derivatives, and in the case of chiral active ingredients it is possible to employ both optically active isomers and racemates or mixtures of diastereoisomers. If desired, the compositions of the invention may also comprise two or more active pharmaceutical ingredients.

Excipients

Examples of excipients which can be added to the dropping liquid are plasticizers. Mention should also be made of thickeners, e.g. colloidal silica, water-soluble cellulose derivatives, acrylates, polyvinylpyrrolidone types and solubilizers such as, for example, polyethylene glycols.

Method Step b)

Delivering droplets of the dispersion comprising the active pharmaceutical ingredient and the copolymer into an aqueous polymer solution which comprises an anionic copolymer incompatible with the cationic copolymer, resulting in precipitation of active ingredient-containing pellets.

Delivering Droplets of the Dispersion

Droplets of the polymer dispersion are delivered by employing a dropping apparatus which brings about a breaking up of a jet of the liquid polymer dispersion. Possible examples thereof are a mechanical chopper, a rotating perforated disk or a vibrating nozzle. Suitable apparatuses are also described for example in Graf, E. and Bothe W. “Mikroverkapselung durch Zertropfen” (Pharmazie in unserer Zeit, 1984, No. 3, pp. 71 to 82).

An appropriate apparatus may be designed for example in such a way that a frequency generator controls a vibrator which causes axial vibrations of a nozzle. The internal diameter of the nozzle may be, for example, 0.1 to 0.5 mm. The frequency of the vibrations may be for example in the range from 5 Hz to 5 kHz, preferably from 20 to 100 Hz, with amplitude being continuously variable in the range from 0 to 3 mm.

The drop-formation process can be monitored by measuring the drop-formation rate. It is then possible to adjust the frequency of the so-called interfering vibration so that the liquid jet vibrates in resonance. Disintegration of the jet can be monitored by employing a light-measurement method with which both the drop-formation rate and the size of the drops can be measured.

The drop-formation rate can be measured using a rate meter as the number of electrical impulses per unit time. If the drop-formation rate and the frequency of the interfering vibration are of equal magnitude, the liquid jet vibrates in resonance, and drops of the same size are formed. The signals can be presented on an oscilloscope or be monitored with the aid of photoelectric detectors or stroboscopes.

The system can be controlled by a control circuit which automatically undertakes appropriate corrections if there are deviations from preset specifications, and thus keeps the actual values within certain tolerances. It is also possible according to the invention to employ for the process concentric multiple nozzles in which the inner drop liquid differs from the outer one. In this case, only the outer drop liquid needs to be incompatible with the polymer in the precipitation bath. Pellets with a core/shell structure are likewise produced.

A further principle suitable for delivering droplets of the liquid is the JETCUTTING® method of GeniaLab® Biotechnologie Produkte und Dienstleistungen GmbH, D-38116 Braunschweig.

Aqueous Polymer Solution

The aqueous polymer solution comprises an anionic copolymer which is incompatible with the cationic copolymer. The aqueous polymer solution may also be referred to as precipitation bath for simplicity.

The anionic copolymer preferably consists of free-radical polymerized units of C₁- to C₄-alkyl ester of acrylic or methacrylic acid and units of (meth)acrylate monomers with carboxyl group radicals.

The (meth)acrylate copolymer consists of from 25 to 95, preferably from 45 to 90, in particular from 45 to 55, % by weight of free-radical polymerized C₁- to C₄-alkyl esters of acrylic or methacrylic acid and may comprise 5 to 75, preferably 10 to 60, in particular 40 to 60, % by weight of (meth)acrylate monomers with an anionic group in the alkyl radical.

C₁- to C₄-alkyl esters of acrylic or methacrylic acid are, in particular, methyl methacrylate, ethyl methacrylate, butyl methacrylate, methyl acrylate, ethyl acrylate and butyl acrylate.

A (meth)acrylate monomer with an anionic group in the alkyl radical may be, for example, acrylic acid, but preferably methacrylic acid.

Suitable anionic (meth)acrylate copolymers are composed of 40 to 60% by weight of methacrylic acid and 60 to 40% by weight of methyl methacrylate or 60 to 40% by weight of ethyl acrylate (EUDRAGIT® L or EUDRAGIT® L100-55 types).

Equally suitable are anionic (meth)acrylate copolymers composed of 20 to 40% by weight of methacrylic acid and 80 to 60% by weight of methyl methacrylate (EUDRAGIT® S type).

Equally suitable are (meth)acrylate copolymers consisting of 10 to 30% by weight of methyl methacrylate, 50 to 70% by weight of methyl acrylate and 5 to 15% by weight of methacrylic acid (EUDRAGIT® FS type).

The copolymers are obtained in a manner known per se by free-radical bulk, solution, bead or emulsion polymerization. They must before processing be brought to the particle size range of the invention by suitable grinding, drying or spraying processes. This can take place by simple crushing of extruded and cooled pellets or hot shot.

The use of powders may be advantageous especially on mixture with other powders or liquids. Suitable apparatuses for producing powders are familiar to the skilled worker, e.g. air jet mills, pinned disk mills, compartment mills. It is possible where appropriate to include appropriate sieving steps. A suitable mill for industrial large quantities is, for example, an opposed jet mill (Multi No. 4200) which is operated with a gage pressure of about 6 bar.

Also suitable are anionic vinyl copolymers and cellulose derivatives, such as crotonic acid copolymers, polyvinyl acetate phthalate (PVAP), cellulose acetate phthalate (CAP), cellulose acetate succinate (CAS), cellulose acetate trimellitate (CAT), hydroxypropylmethylcellulose phthalate (HPMCP) and/or carboxymethylcellulose (CMEC).

It is also possible according to the invention to employ mixtures of these polymers in the precipitation bath.

Partial or complete neutralization of the acid groups is usually necessary in order to prepare a solution of the anionic copolymer. The anionic copolymer may for example be stirred in small portions into water in a final concentration of 1 to 40% by weight and, at the same time, be partially or completely neutralized by adding a basic substance such as, for example, NaOH. It is also possible to employ a powder of an anionic copolymer to which a base, e.g. NaOH, has been added during its preparation for the purpose of (partial) neutralization, so that the powder is already a (partially) neutralized polymer. The pH of the solution is usually above 4.

The viscosity of the aqueous polymer solution is preferably about the same as or, particularly preferably, lower than the viscosity of the polymer dispersion and can be for example 10 to 1000 mPa s (measured in accordance with DIN/ISO ?).

The aqueous polymer solution can be adjusted to a temperature below room temperature, e.g. 5, 10 or 20° C. lower. The temperature of the aqueous polymer solution may be for example from 4 to 25° C. A low temperature is favorable for preventing agglomeration.

Excipients

Excipients which can be added to the precipitation bath are, for example, osmotically active electrolytes which expedite the hardening of the spherically coagulated particles and thus promote uniform shaping. Mention should further be made of release agents which prevent adhesion of the particles. Examples are lipophilic emulsifiers with an HLB below 7 or conventional mold release agents such as silicones, talc, magnesium stearate, ground silica or kaolin.

Method Step c)

Removing the active ingredient-containing pellets from the aqueous polymer solution and subsequently drying the pellets.

The precipitated pellets can be collected for example on sieves, expediently exchangeable sieves. The drying can take place by conventional methods, e.g. in trays or in the fluidized bed of a fluidized bed generator.

The temperature during this should be below the glass transition temperature Tg of the methacrylate copolymers used in order to prevent adhesion of the pellets. The drying can take place for example at 30 to 50° C. in a circulating air drying oven for 24 to 72 hours.

Active Ingredient-containing Pellets with Multilayer Core/Shell Structure

The active ingredient-containing pellets obtainable by the method of the invention have a multilayer core/shell structure. In this case, the active ingredient is formulated in the core together with the cationic copolymer, followed by a transitional zone in which a transition zone of active ingredient cationic copolymer and anionic copolymer mixed therewith follows transitionally, and is finally enclosed by an outer shell with the anionic copolymer.

The active ingredient content decreases from the core into the transitional zone and may moreover for example transitionally undergo a transition from the particulate form (solid dispersion) into a dissolved form (solid solution) with higher bioavailability. Differentiation or detection of the two states is possible for example by DSC (differential scanning calorimetry) or by X-ray diffraction analysis.

The particles may be round or slightly ellipsoidal and have a size or diameter in the range from 100 to 2000 μm.

Compared with a conventional bilayer structure of active ingredient-containing pellets as disclosed for example in DE 100 13 029, it is possible to obtain other, delayed release profiles, to which on the one hand the particle size, but especially the transitional zone, contributes. Further advantageous properties of the pellets of the invention are taste and odor masking or improved bioavailability of slightly soluble active ingredients.

Pharmaceutical Forms

The pellets produced according to the invention can be processed to pharmaceutical forms by packing into capsules or sachets, by compression to tablets with accelerated or delayed disintegration, as constituent of powders for reconstitution, as base material for embeddings in other pharmaceutical forms such as suppositories, forms for vaginal use, implants, films or dermatologicals such as, for example, transdermal therapeutic systems. Further areas of use which should be mentioned are diagnostic aids or cosmetics.

### EXAMPLES

EUDRAGIT® RS 30D: 30% strength dispersion comprising a copolymer of 65% by weight of methyl methacrylate, 30% by weight of ethyl acrylate and 5% by weight of 2-trimethylammoniumethyl methacrylate chloride.

EUDRAGIT® L 100-55: copolymer of 50% by weight of methacrylic acid and 50% by weight of ethyl acrylate.

### Example 1

### Slow-release Propranol Pellets

a) Dropping Dispersion:

| | | | % by weight |
|---|---|---|---|
| | % by | Dry matter | in the dry |
| Ingredients | weight | (% by weight) | matter |
| EUDRAGIT ® RS 30 D | 89.0 | 26.7 | 71.8 |
| Polysorbate 80 | 0.4 | 0.4 | 1.1 |
| Acetyl tributyl citrate | 4.0 | 4.0 | 10.8 |
| HCl (10% strength) | 0.7 | 0.1 | 0.2 |
| Propranolol HCl | 6.0 | 6.0 | 16.1 |
| Total | 100.0 | 37.2 | 100.0 |

Polysorbate 80 and acetyl tributyl citrate are homogenized using a stirrer (e.g. magnetic stirrer. The EUDRAGIT RS 30 D is incorporated using a high-speed stirrer (e.g. Ultra Turrax). The dispersion is adjusted to pH 3.5 with 10% strength HCl, and finally, after addition of propranolol HCl, homogenized with the Ultra Turrax for a further 10 min.

b) Precipitation Bath

| | | | % by weight |
|---|---|---|---|
| | % by | | in the dry |
| Ingredients | weight | Dry matter | matter |
| EUDRAGIT ® L100-55 | 5.00 | 5.0 | 38.5 |
| 20% by weight sodium | 2.50 | 0.5 | 3.8 |
| hydroxide solution, | | | |
| aqueous, 45% partially | | | |
| neutralized with NaOH | | | |
| 20% strength NaCl solution, | | 7.5 | 57.7 |
| aqueous | 37.50 | | |
| Purified water | 55.00 | — | — |
| Total | 100.00 | 13.0 | 100.0 |

EUDRAGIT L 100-55 is dispersed (added in portions) in the water using a paddle stirrer. The solution resulting after addition of the sodium hydroxide solution and the NaCl solution is stirred for 1 hour.

c) Dropping Conditions:

V=148 g of dispersion/h (delivery: eccentric screw pump)

Vibration frequency at the nozzle during the delivery of droplets: 50 Hz

Internal diameter of nozzle: 0.3 mm

d) Separation and Drying

Separation by filter apparatus (see drawing).

Drying 40° C. for 48 hours (circulating air drying oven)

e) Data for the Pellets:

Shape: round to slightly ellipsoidal, solid, 62% active ingredient content (analyzed by UV photometry)

Sieve analysis:

| >2.0 mm | 2.3% (mass) |
|---|---|
| 2.0-1.4 mm | 30.1% |
| 1.4-1.0 mm | 57.1% |
| 1.0-0.8 mm | 9.8% |
| 0.8-0.5 mm | 0.4% |
| <0.5 mm | 0.3% |

Analytical results:

Active ingredient release (paddle 100 rpm) 2 h, pH 1.0 then changed to pH 6.8:

Active ingredient content: 10.0% (=62.0% of theory)

| | % release of |
|---|---|
| [Time h] | theory |
| 0.5 | 59.7 |
| 1 | 52.5 |
| 2 | 96.0 |
| 3 | 96.1 |
| 4 | 98.9 |
| 6 | 99.4 |

### Example 2

### Slow-release Verapamil Pellets (VB 89/55)

a) Dropping Dispersion:

| | | | % by weight |
|---|---|---|---|
| | % by | Dry matter | in the dry |
| Ingredients | weight | (% by weight) | matter |
| EUDRAGIT ® RS 30 D | 91.8 | 27.5 | 77.0 |
| Polysorbate 80 | 1.8 | 1.8 | 5.1 |
| Acetyl tributyl citrate | 3.0 | 3.0 | 8.4 |
| HCl (10% strength) | 0.7 | 0.1 | 0.3 |
| Verapamil HCl | 3.3 | 3.3 | 9.2 |
| Total | 100.0 | 35.7 | 100.0 |

Polysorbate 80 and acetyl tributyl citrate are homogenized using a stirrer (e.g. magnetic stirrer. The EUDRAGIT RS 30 D is incorporated using a high-speed stirrer (e.g. Ultra Turrax). The dispersion is adjusted to pH 3.5 with 10% strength HCl, and finally, after addition of verapamil HCl, homogenized with the Ultra Turrax for a further 10 min.

b) Precipitation Bath Analogous to Example 1

c) Dropping Conditions:

V=239 g of dispersion/h (delivery: eccentric screw pump)

Vibration frequency at the nozzle delivery of droplets: 50 Hz

Internal diameter of nozzle: 0.3 mm

d) Separation and Drying

Separation by filter apparatus (see drawing).

Drying 40° C. for 48 hours (circulating air drying oven)

e) Data for the Pellets:

Shape: round to slightly ellipsoidal, solid,

Sieve analysis:

| >2.0 mm | 19.4% (mass) |
|---|---|
| 2.0-1.0 mm | 52.2% |
| 1.0-0.5 mm | 26.9% |
| <0.5 mm | 1.5% |

Analytical results:

Active ingredient content: 7.44% (=80.6% of theory)

Active ingredient release (matrix beads 1.0-2.0 mm; paddle 100 rpm) 2 h, pH 1.0 then changed to pH 6.8:

| | % release of the | % release of the | % release of the |
|---|---|---|---|
| | 0.50-1.00 mm | 1.00-2.00 mm | 2.00-3.2 mm |
| [Time h] | fraction | fraction | fraction |
| 0 | 0 | 0 | 0 |
| 2 | 94 | 75.9 | 53 |
| 3 | 95 | 82.0 | 56 |
| 4 | 95.8 | 83.3 | 57 |
| 6 | 96.5 | 87.1 | 58 |
| 8 | 99 | 88.2 | 61 |
| Homogenized | 100.0 | 100.0 | 100 |

### Example 3

### Slow-release Propranolol Pellets with Thickened Dipping Dispersion

a) Dropping Dispersion:

| | | | % by weight |
|---|---|---|---|
| | % by | Dry matter | in the dry |
| Ingredients | weight | (% by weight) | matter |
| EUDRAGIT ® RS 30 D | 92.0 | 27.6 | 77.5 |
| Propranolol HCl | 6.2 | 6.2 | 17.4 |
| Aerosil ® 200 | 1.8 | 1.8 | 5.1 |
| Total | 100.0 | 35.6 | 100.0 |

EUDRAGIT RS 30 D is introduced, and propranolol HCl and Aerosil 200 are successively dispersed in the dispersion using the Ultra Turrax (dispersion time 10 minutes in each case).

b) Precipitation Bath Analogous to Example 1

c) Dropping Conditions:

V=283 g of dispersion/h (delivery: eccentric screw pump)

Vibration frequency at the nozzle during the delivery of droplets: 50 Hz

Internal diameter of nozzle: 0.3 mm

d) Separation and Drying

Separation by filtration apparatus (see drawing).

Drying 40° C. for 48 hours (circulating air drying oven)

e) Data for the pellets: Shape: round to slightly ellipsoidal, solid.

1: A method for the production of active ingredient-containing pellets comprising
a) suspending or dissolving an active pharmaceutical ingredient in a dispersion which comprises a cationic copolymer
b) delivering droplets of the dispersion comprising the active pharmaceutical ingredient and the copolymer into an aqueous polymer solution which comprises an anionic copolymer incompatible with the cationic copolymer, resulting in precipitation of active ingredient-containing pellets
c) removal of the active ingredient-containing pellets from the aqueous polymer solution and subsequent drying of the pellets.

2: The method as claimed in claim 1, wherein the cationic copolymer consists of free-radical polymerized units of C₁- to C₄-alkyl esters of acrylic or methacrylic acid and units of (meth)acrylate monomers with tertiary or quaternary amino or ammonium groups.

3: The method as claimed in claim 1 wherein the anionic copolymer consists of free-radical polymerized units of C₁- to C₄-alkyl esters of acrylic or methacrylic acid and units of (meth)acrylate monomers with carboxyl group radicals.

4: The method as claimed in claim 1 wherein the viscosity of the polymer dispersion is higher than the viscosity of the aqueous polymer solution.

5: The method as claimed in claim 4, wherein the viscosity of the polymer dispersion measured by method 2.2.10 to Pharm. Eur. 3^{rd} edition is from 20 to 5 000 mPa s and the viscosity of the aqueous polymer solution is from 10 to 1 000 mPa s.

6: The method as claimed in claim 1, wherein the polymer dispersion and/or the aqueous polymer solution are adjusted to a temperature below room temperature.

7: The method as claimed in claim 1, wherein a dropping apparatus which breaks up a liquid jet of the polymer dispersion is employed for the delivery of droplets of the polymer dispersion.

8: Active ingredient-containing pellets with a multilayer core/shell structure which can be produced in a method as claimed in claim 1.

9: A pharmaceutical form, comprising pellets as claimed in claim 8.

10: The pharmaceutical form as claimed in claim 9, in the form of capsules, sachets, tablets with accelerated or delayed disintegration, powders for reconstitution, suppositories, products for vaginal use, implants, films or dermatologicals such as, for example, transdermal therapeutic systems.

11: The use of active ingredient-containing pellets as claimed in claim 8 in diagnostic aids or cosmetics.
